(19) United States
(12) Patent Application Publication
(10) Pub. No.: US 2025/0098968 A1
(43) Pub. Date: Mar. 27, 2025

(54) PHYSIOLOGICAL DETECTION DEVICE AND WEARABLE DEVICE WITH REDUCED MOTION ARTIFACT

(71) Applicant: PIXART IMAGING INC., Hsin-Chu County (TW)

(72) Inventors: Ren-Hau GU, Hsin-Chu County (TW); Hsiu-Ling YEH, Hsin-Chu County (TW)

(21) Appl. No.: 18/473,429

(22) Filed: Sep. 25, 2023

(51) Int. Cl. A61B 5/024; A61B 5/00

(52) U.S. Cl. CPC A61B 5/02433; A61B 5/02438; A61B 5/7214; A61B 2562/0219

(57) ABSTRACT

There is provided a physiological detection device for detecting physiological signals via a skin surface and including a first light source, a second light source, a first light sensor, a second light sensor and a processor. The first light source emits light via an optical element that causes the first light sensor and the second light sensor to receive different percentages of light energy of the first light source. The first light sensor and the second light sensor receive the same percentage of light energy of the second light source. The processor adjusts emission intensity of the first light source according to an intensity difference between two light energies received by the first and second light sensors as well as an intensity variation of light energy received by the first light sensor to alleviate motion artifacts in the physiological signals.

### FIELD OF THE DISCLOSURE

This disclosure generally relates to a physiological detection device and, more particularly, to a physiological detection device using multiple light sources of different wavelengths and multiple light sensors to eliminate motion artifacts and a wearable device using the same.

### BACKGROUND OF THE DISCLOSURE

The physiological signal, e.g., including photoplethysmography (PPG) and oxygen saturation SPO₂, detected by an optical device has been applied to various applications such as identifying a heart rate or an identity of a user. Meanwhile, because the optical device being used has a small size and low power consumption, it can be easily applied to wearable devices.

However, the wearable device will have a relative movement with respect to a detected skin surface when a user has a motion such that motion artifacts are generated in optical detection signals. The motion artifacts form noises to degrade the detection accuracy.

Presently, to eliminate the motion artifact, an accelerometer or a Gyro is further arranged on the wearable device at the same time to provide information associated with the user's motion. By comparing the motion information with the optical detection signals, it is possible to remove motion induced signals from the optical detection signals. However, under continuous motions or low temperatures, the motion artifact cannot be effectively removed by adopting the accelerometer or the Gyro such that the detection accuracy is not significantly improved.

Accordingly, the present disclosure further provides a physiological detection device that reduces motion artifacts in an optical phase, and an electronic device using the same.

### SUMMARY

The present disclosure provides an optical physiological detection device that adopts multiple light sources of different wavelengths and multiple light sensors. The multiple light sources include at least two wavelengths that respectively have a high responsivity and a low responsivity to motions so as to reduce interference from motion artifacts directly in an analog stage.

The present disclosure further provides an optical physiological detection device that is adapted to a wearable device or a portable device in which no additional accelerometer or Gyro is required so as to reduce the system complexity and cost.

The present disclosure provides a physiological detection device including a first light source, a second light source, a first light sensor, a second light sensor and a processor. The first light source is configured to emit light toward a skin surface via an optical element. The second light source is configured to emit light toward the skin surface. The first light sensor is configured to receive a first intensity light associated with the first light source and a second intensity light associated with the second light source from the skin surface to generate a first detection data. The second light sensor is configured to receive a third intensity light associated with the first light source and the second intensity light associated with the second light source from the skin surface to generate a second detection data, wherein the optical element causes the third intensity light to be lower than the first intensity light. The processor is configured to adjust a first emission intensity of the first light source and fix a second emission intensity of the second light source.

The present disclosure further provides a physiological detection device including a first light source, a second light source, a first light sensor, a second light sensor and a processor. The first light source is configured to emit light toward a skin surface. The second light source is configured to emit light toward the skin surface. The first light sensor is configured to receive outgoing light from the skin surface to generate a first detection data. The second light sensor is configured to receive outgoing light from the skin surface to generate a second detection data. The first light source, the second light source, the first light sensor and the second light sensor are arranged to cause the first light sensor and the second light sensor to receive different intensity of the outgoing light associated with the first light source, and cause the first light sensor and the second light sensor to receive identical intensity of the outgoing light associated with the second light source.

The present disclosure further provides an operating method of a wearable device. The wearable device is attached to a skin surface and includes an infrared light source, a green light source, a first light sensor, a second light sensor and a processor. The operating method includes the steps of: lighting the infrared light source and the green light source; detecting, using the first light sensor, a first intensity light of the infrared light source and a second intensity light of the green light source to generate a first detection data; detecting, using the second light sensor, a third intensity light of the infrared light source and the second intensity light of the green light source to generate a second detection data, wherein the third intensity light is lower than the first intensity light; and adjusting, using the processor, an emission intensity of the infrared light source according to an intensity difference between the first detection data and the second detection data as well as an intensity variation of the first detection data.

### BRIEF DESCRIPTION OF DRAWINGS

Other objects, advantages, and novel features of the present disclosure will become more apparent from the following detailed description when taken in conjunction with the accompanying drawings.

FIG. 1 is a schematic diagram of an arrangement of light sources and light sensors of a physiological detection device with respect to a skin surface according to one embodiment of the present disclosure.

FIG. 2 is a top view of an arrangement of light sources and light sensors of a physiological detection device according to one embodiment of the present disclosure.

FIG. 3 is another top view of an arrangement of light sources and light

sensors of a physiological detection device according to one embodiment of the present disclosure.

FIG. 4 is a side view of an arrangement of light sources and light sensors of a physiological detection device according to one embodiment of the present disclosure.

FIG. 5A is a schematic diagram of the absolute intensity difference of a physiological detection device according to one embodiment of the present disclosure.

FIGS. 5B and 5C are schematic diagrams of the first detection data

changes with light source intensity adjustment of a physiological detection device according to one embodiment of the present disclosure.

FIG. 6 is a diagram of a detected light signal, without being denoised by adjusting light source intensity, of a first light sensor of a physiological detection device according to one embodiment of the present disclosure.

FIG. 7 is a diagram of a detected light signal, denoised by adjusting light source intensity, of a first light sensor of a physiological detection device according to one embodiment of the present disclosure.

FIG. 8 is a flow chart of an operating method of a wearable device according to one embodiment of the present disclosure.

### DETAILED DESCRIPTION OF THE DISCLOSURE

It should be noted that, wherever possible, the same reference numbers will be used throughout the drawings to refer to the same or like parts.

One objective of the present disclosure is to provide a physiological detection device including multiple light sources of different optical wavelengths and multiple light sensors. The multiple optical wavelengths include at least infrared light and green light, but not limited to. The physiological detection device is used to detect physiological signals, e.g., including PPG signals and SPO₂, via a skin surface. The PPG signals may be used to detect a heart rate, a breath rate and a blood pressure of a user, which is known to the art and not a main objective of the present disclosure, and thus details thereof are not described herein. The objective of the present disclosure is to alleviate motion artifacts in physiological signals by using an optical technique.

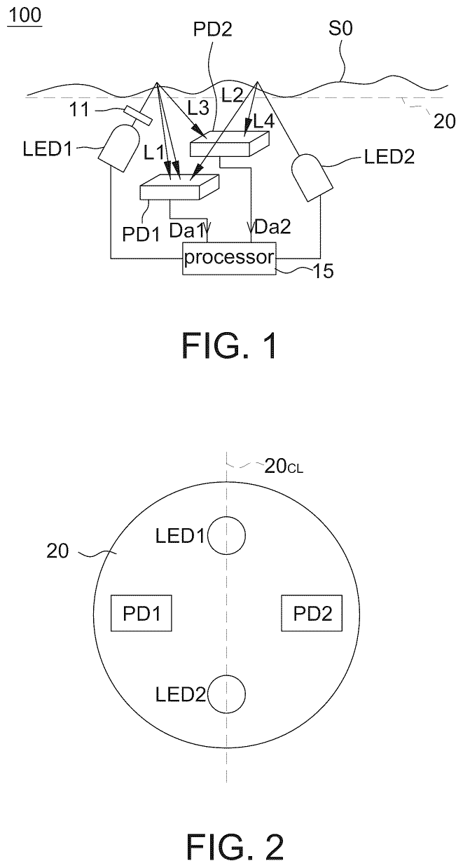

Please refer to FIG. 1, it is a schematic diagram of an arrangement of light sources and light sensors of a physiological detection device 100 with respect to a skin surface S0 according to one embodiment of the present disclosure. The physiological detection device 100 is arranged on, for example, a portable electronic device or a wearable electronic device without particular limitations as long as the electronic device has a case surface (e.g., the element 20 shown in FIGS. 1 to 4) in contact with the skin surface S0 of a user. For example, when the electronic device is a watch or a bracelet, the case surface 20 is an inner surface of the watch or the bracelet.

Please refer to FIGS. 1 and 2, FIG. 2 is a top view of a case surface 20 of a physiological detection device 100 according to one embodiment of the present disclosure, which shows a two-dimensional position arrangement between elements. The physiological detection device 100 includes a first light source LED1, a second light source LED2, a first light sensor PD1, a second light sensor PD2, an optical element 11 and a processor 15. These elements are all arranged under the case surface 20, and the case surface 20 preferably has multiple windows (e.g., referring to FIG. 4) respectively opposite to the first light source LED1, the second light source LED2, the first light sensor PD1 and the second light sensor PD2 to allow lights to penetrate therethrough. In another aspect, the case surface 20 has a single window allow light to go out from the first and second light sources and impinge onto the first and second light sensors therethrough.

In one aspect, the first light source LED1 preferably emits light of an optical wavelength more sensitive to detecting a motion artifact, e.g., red light source and/or infrared light source. The second light source LED2 preferably emits light of an optical wavelength more sensitive to detecting a PPG signal but less sensitive to detecting the motion artifact, e.g., green light source. In the present disclosure, the first light source LED1 and the second light source LED2 preferably emit light simultaneously such that the first light sensor PD1 and the second light sensor PD2 detect light energy associated with both the first light source LED1 and the second light source LED2. As shown in FIG. 1, said light energy is energy of reflection light from the skin surface S0.

It should be mentioned that although the present disclosure is illustrated by a reflective physiological detection device, the present disclosure is not limited thereto. In other aspects, the physiological detection device is a transmissive physiological detection device, which may be referred to U.S. application Ser. No. 17/462,622, entitled “HEART RATE DETECTING DEVICE CAPABLE OF ALLEVIATING MOTION INTERFERENCE” filed on Aug. 31, 2021, assigned to the same assignee of the present application, and the full disclosure of which is incorporated herein by reference.

In one aspect, the optical element 11 is, for example, a view control film (VCF) for controlling light penetrates therethrough to have different light intensity in different angles (e.g., incident angle or emergent angle), but not limited to. In another aspect, the optical element 11 may be a customized lens. In a further aspect, by arranging a light guiding structure of the first light source LED1, the same effect can be realized as arranging the optical element 11. That is, the optical element 11 is a device separately manufactured from the first light source LED1, or a structure integrated with the first light source LED1 without particular limitations.

The first light source LED1 emits light with a first emission intensity toward a skin surface S0 via the optical element 11. The second light source LED2 emits light with a second emission intensity toward the skin surface S0. Since a main purpose of arranging the first light source LED1 is to detect motion artifacts, in one aspect the first emission intensity is preferably smaller than 10% of the second emission intensity. In one aspect, the second light source LED2 emits light via another optical element (e.g., a lens), but said another optical element does not distribute different light energy to different views/angles. In another aspect, according to a different spatial arrangement (e.g., examples given below), another view control film (e.g., VCF2) is arranged in a light path of emission light of the second light source LED2 to cause the first light source PD1 and the second light source LED2 to receive substantially identical light intensity through the VCF2.

The first light sensor PD1 receives a first intensity light of outgoing light (e.g., shown as L1 FIG. 1) associated with the first light source LED1 and a second intensity light of outgoing light (e.g., shown as L2 in FIG. 1) associated with the second light source LED2 to generate a first detection data Da1 (e.g., a gray level value). That is, the first detection data Da1 is a response summation of the outgoing light L1 and L2.

The second light sensor PD2 receives a third intensity light of outgoing light (e.g., shown as L3 FIG. 1) associated with the first light source LED1 and a fourth intensity light of outgoing light (e.g., in FIG. 1 shown as L4, which normally has substantially identical light intensity to L2) associated with the second light source LED2 to generate a second detection data Da2 (e.g., a gray level value). That is, the second detection data Da2 is a response summation of the outgoing light L3 and L4.

In the present disclosure, the optical element 11 causes the third intensity light to be lower than the first intensity light. In one aspect, the first intensity light is more than two times of the third intensity light, but the present disclosure is not limited thereto as long as the first intensity light is clearly larger than the third intensity light.

Please refer to FIG. 2 again, since this aspect is to distribute different percentages of outgoing light associated with the first light source LED1 received by the first light sensor PD1 and the second light sensor PD2 using the optical element 11, the first light source LED1 is arranged at a center line 20_{CL} between the first light sensor PD1 and the second light sensor PD2. The second light source LED2 is also arranged at the center line 20_{CL} such that the first light sensor PD1 and the second light sensor PD2 receive the same percentage of outgoing light associated with the second light source LED2. In this case, the VCF2 (if there is) distributes identical light intensity to the first light sensor PD1 and the second light sensor PD2.

The processor 15 is, for example, a micro controller unit (MCU), an application specific integrated circuit (ASIC) or a field programmable gate array (FPGA), which performs operations and controls thereof using software, hardware and/or firmware. For example, the processor 15 is electrically coupled to the first light source LED1, the second light source LED2, the first light sensor PD1 and the second light sensor PD2 to control light emission of the light sources and to receive detection data (e.g., gray level values) of the light sensors. In one aspect, the processor 15 adjusts emission intensity of one of the first light source LED1 and the second light source LED2, e.g., adjusting the first emission intensity of the first light source LED1 and fixing the second emission intensity of the second light source LED2, but not limited thereto.

For example, the processor 15 determines an adjustment of the first emission intensity according to an absolute value of an intensity difference |Da1−Da2| between the first detection data Da1 and the second detection data Da2. As mentioned above, since the second emission intensity of the second light source LED2 (mainly contribute to PPG signals) is fixed in one example mentioned therein, information associated with the PPG signal is substantially cancelled by calculating the intensity difference. The magnitude of the intensity difference (i.e. |Da1−Da2|) reflects a value of noises (i.e. motion artifacts). In one aspect, when the absolute intensity difference |Da1−Da2| is larger, a larger adjustment is selected.

For example, the processor 15 further determines an adjusting direction of the first emission intensity according to an intensity variation of the first detection data Da1. As mentioned above, since the second emission intensity of the second light source LED2 is fixed, the intensity variation of the first detection data Da1 is considered to be caused by a change of noises (i.e. motion artifacts).

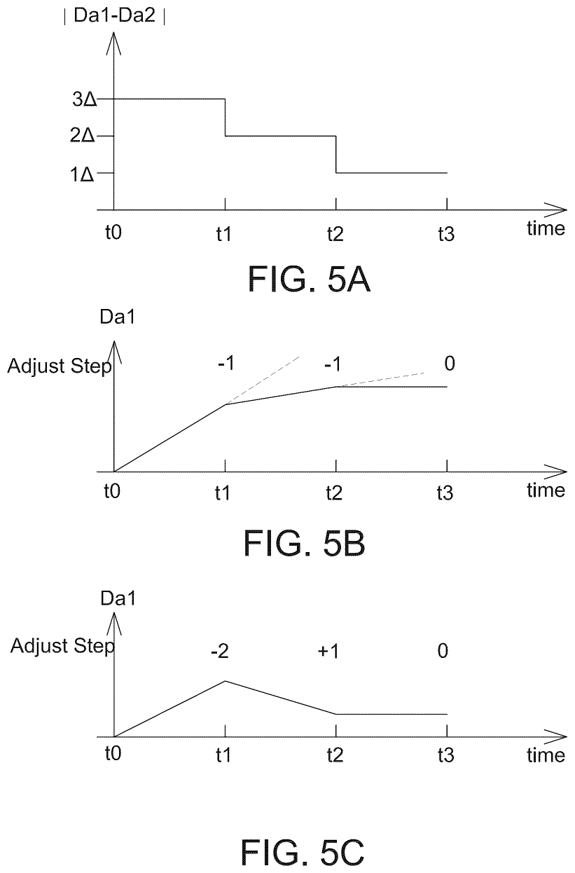

Please refer to FIGS. 5A to 5C, they are schematic diagrams of an operating method of adjusting the first light source LED1 (e.g., shown as Adjust Step) according to the absolute intensity difference |Da1−Da2| and an intensity variation (e.g., slop) of the first detection data Da1. Since the outgoing light L1 and L3 have different light intensity, the first detection data Da1 and the second detection data Da2 have a basic difference, e.g., shown as 14. When the absolute intensity difference |Da1−Da2| is equal to 14, the processor 15 does not adjust the first light source LED1, i.e. the Adjust Step is 0.

In FIGS. 5A to 5C, it is assumed that the processor 15 obtains |Da1−Da2|=3Δ between times t0 and t1, and the intensity variation (obtained by calculating a difference between first detection data at t1 and t0) of the first detection data Da1 is increasing, which means noises increase a value of the first detection data Da1. In this case, the processor 15 adjusts the first emission intensity of the first light source LED1 downward by one step (e.g., FIG. 5B showing the Adjust Step=−1) or adjusts the first emission intensity of the first light source LED1 downward by two steps (e.g., FIG. 5C showing the Adjust Step=−2).

After the adjustment, the processor 15 obtains |Da1−Da2|=2Δ between times t1 and t2, and the intensity variation (obtained by calculating a difference between first detection data at t2 and t1) of the first detection data Da1 is increasing (FIG. 5B) or decreasing (FIG. 5C). In this case, the processor 15 adjusts the first emission intensity of the first light source LED1 downward by one step (e.g., FIG. 5B showing the Adjust Step=−1) or adjusts the first emission intensity of the first light source LED1 upward by one step (e.g., FIG. 5C showing the Adjust Step=+1)

In the present disclosure, an Adjust Step corresponding to the calculated |Da1−Da2| is preset before shipment.

After the adjustment, the processor 15 obtains |Da1−Da2|=1Δ between times t2 and t3, and the intensity variation (obtained by calculating a difference between first detection data at t3 and t2) of the first detection data Da1 is substantially unchanged, and thus the processor 15 stops adjusting the first emission intensity of the first light source LED1.

In one aspect, when the intensity variation of the first detection data Da1 is substantially not changed between t2 and t3, the first emission intensity of the first light source LED1 is not adjusted even when |Da1−Da2|>1Δ. In another aspect, when|Da1−Da2|=1Δ, the first emission intensity of the first light source LED1 is not adjusted even when the intensity variation of the first detection data Da1 is still changing (i.e. increasing or decreasing). In other words, the first emission intensity of the first light source LED1 is adjusted only when the first detection data Da1 has an intensity variation and |Da1−Da2|>1Δ.

More specifically, when the intensity variation of the first detection data Da1 is larger than or equal to a positive threshold (e.g., slope larger than or equal to a predetermined threshold), the processor 15 decreases the first emission intensity of the first light source LED1 by the adjustment, which is determined according to |Da1−Da2|; when the intensity variation of the first detection data Da1 is smaller than or equal to a negative threshold (e.g., slope smaller than or equal to a predetermined threshold), the processor 15 increases the first emission intensity of the first light source LED1 by the adjustment, which is determined according to |Da1−Da2|; whereas, when the intensity variation of the first detection data Da1 is between the positive threshold and the negative threshold or not changing, the processor 15 stops adjusting the first emission intensity of the first light source LED1, indicating converged.

For example, the physiological detection device 100 further includes a memory for previously recording adjustments of the first emission intensity (e.g., the Adjust Step mentioned above) corresponding to different values of |Da1−Da2| for being accessed by the processor 15.

It should be mentioned that values and variations in FIGS. 5A to 5C are only intended to illustrate but not to limit the present disclosure. During operations, user's motion can cause |Da1−Da2| to change randomly.

In another aspect, the processor 15 multiplies Da2 by a multiple RA to cause RA×Da2=Da1 when there is no motion, i.e. |Da1−RA×Da2|=0. In this case, when a motion occurs, |Da1−RA×Da2| becomes larger than 0. This can also be shown by subtracting 1Δ from longitudinal values in FIG. 5A. In other words, in this aspect, the first emission intensity of the first light source LED1 is adjusted only when the intensity variation of the first detection data Da1 occurs and |Da1−RA×Da2|>0.

In the above embodiments, the optical element 11 is arranged to cause light intensity of the outgoing light L1 and L3 to be different, but the present disclosure is not limited thereto. In the present disclosure, the first light sensor PD1 and the second light sensor PD2 are arranged to receive different intensity of the outgoing light L1 and L3 associated with the first light source LED1 and to receive identical intensity of the outgoing light L2 and L4 associated with the second light source LED2 by the spatial arrangement of the first light source LED1, the second light source LED2, the first light sensor PD1 and the second light sensor PD2.

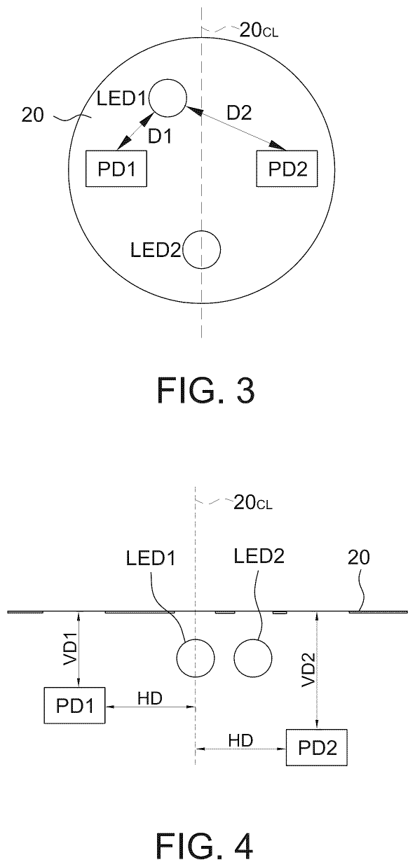

Please refer to FIG. 3, in one aspect, the first light source LED1 is arranged to be closer to (e.g., shown at left side of a center line 20_{CL}) the first light sensor PD1 but farther from the second light sensor PD2 (i.e. D1<D2) so as to realize the purpose of a light intensity of the outgoing light L1 associated with the first light source LED1 received by the first light sensor PD1 is more than two times of a light intensity of the outgoing light L3 associated with the first light source LED1 received by the second light sensor PD2. In this case, the optical element 11 is not adopted to distribute emission light.

Or, an optical element 11 is still adopted to fine tune emission light distribution to insure a predetermined intensity ratio of L1 and L3 is received by the first light sensor PD1 and the second light sensor PD2 to compensate the assembly error of the first light sensor PD1, the second light sensor PD2, the first light source LED1 and the second light source LED2.

Please refer to FIG. 4, in another aspect, the first light source LED1 is arranged at the center line 20_{CL}, but the first light sensor PD1 is closer to the case surface 20 and the second light sensor PD2 is farther from the case surface 20, i.e. VD1<VD2. Meanwhile, to cause the outgoing light L2 and L4 to have identical light intensity, the second light source LED2 is arranged to be closer to (e.g., shown at right side of a center line 20_{CL}) the second light sensor PD2 and farther from the first light sensor PD1. In this case, the optical element 11 is not adopted to distribute emission light.

Or, an optical element 11 is still adopted to fine tune emission light distribution to insure a predetermined intensity ratio of L1 and L3 is received by the first light sensor PD1 and the second light sensor PD2 to compensate the assembly error of the first light sensor PD1, the second light sensor PD2, the first light source LED1 and the second light source LED2.

The spatial arrangement of the first light source LED1, the second light source LED2, the first light sensor PD1 and the second light sensor PD2 is not limited to those shown in in FIG. 3 and FIG. 4. The arrangement of multiple light sources and multiple light sensors is to cause different light sensors to receive different intensity of red/infrared light but receive identical intensity of green light.

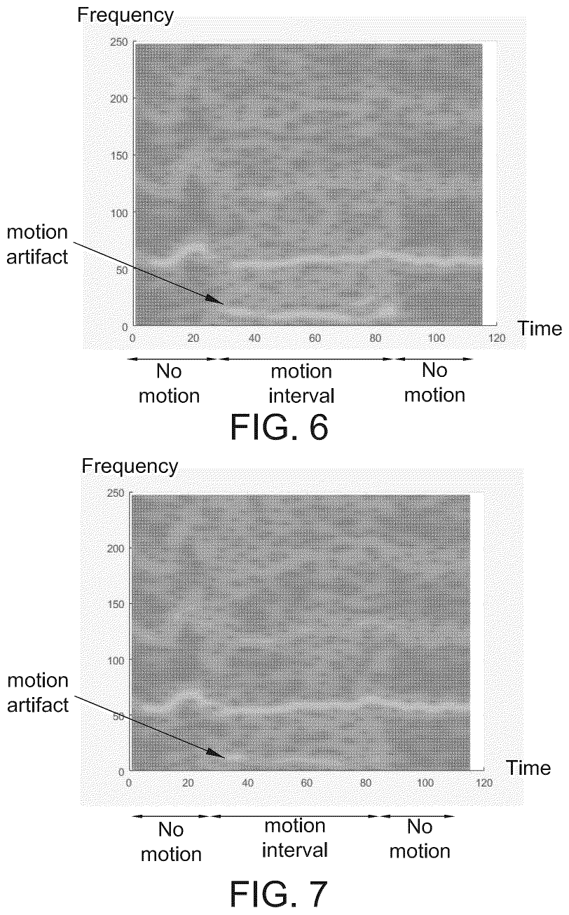

After the intensity variation of the first detection data Da1 is converged (i.e. substantially no change) by regulating the first emission intensity of the first light source LED1, the interference from motion artifacts are considered to be cancelled. For example, FIG. 6 shows that a signal of the motion artifact appears in the first detection data Da1 within a motion interval of the user's skin surface S0. After regulating the first emission intensity of the first light source LED1 (e.g., shown in FIGS. 5A to 5C), the signal of the motion artifact in the first detection data Da1 is effectively reduced as shown in FIG. 7. Then, the processor 15 generates the PPG signal according to the first detection data Da1 (denoised) without according to the second detection data Da2. The applications of the PPG signal are determined according to different requirements without particular limitations.

It should be mentioned that the motion mentioned above is not limited to the skin surface S0 itself but also includes movements of tissues (e.g., muscles) under the skin surface S0.

In another aspect, the processor 15 generates the PPG signal further according to a weighted combination of the first detection data Da1 and the second detection data Da2.

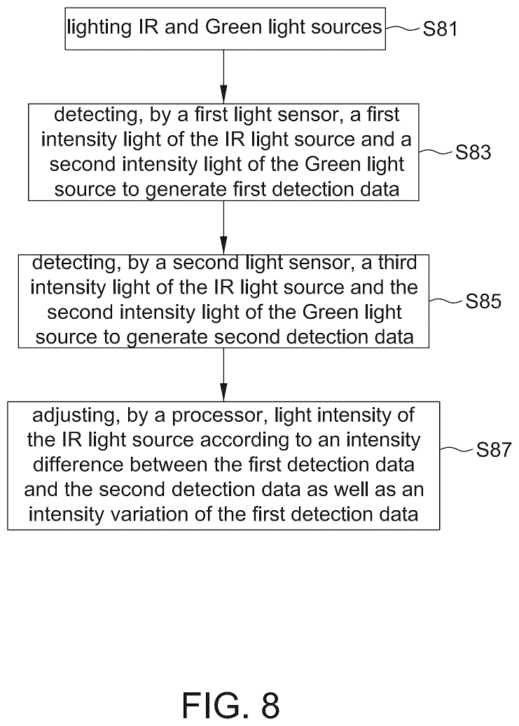

Please refer to FIG. 8, it is an operating method of a wearable electronic device adopting the physiological detection device 100 of the present disclosure, including the steps of: lighting an infrared light source LED1 and a green light source LED2 (Step S81); detecting, using a first light sensor PD1, a first intensity light of the infrared light source LED1 and a second intensity light of the green light source LED2 to generate a first detection data Da1 (Step S83); detecting, using a second light sensor PD2, a third intensity light of the infrared light source LED1 and the second intensity light of the green light source LED2 to generate a second detection data Da2, wherein the third intensity light is lower than the first intensity light (Step S85); and adjusting, using a processor 15, an emission intensity of the infrared light source LED1 according to an intensity difference between the first detection data Da1 and the second detection data Da2 as well as an intensity variation of the first detection data Da1 (Step S87).

Details of this operating method have been illustrated above, e.g., referring to FIG. 5, and thus are not repeated again. The method of adjusting an emission intensity of a light emitting diode is known to the art, e.g., changing drive current thereof, and thus details thereof are not described herein. This operating method of a wearable device is also considered as an operating method of the physiological detection device 100 of the present disclosure.

It should be mentioned that the values mentioned herein are only intended to illustrate but not to limit the present disclosure.

It should be mentioned that the infrared light and green light mentioned herein do not indicate a single optical wavelength but indicate an optical wavelength range.

It should be mentioned that although the above embodiments are illustrated by using light emitting diodes (LED) and photodiodes (PD) respectively as the light sources and light sensors, the present is not limited thereto. In another aspect, the light sources are LEDs and/or laser diodes, and the light sensors are PDs and/or single photon avalanche diodes (SPAD) without particular limitations.

As mentioned above, the conventional wearable devices adopt an additional accelerometer or a Gyro to detect motions of a user, but the denoising effect is not satisfactory in exercising or under low temperatures. Accordingly, the present disclosure further provides a full-optical physiological detection device (e.g., referring to FIGS. 1-4) and an operating method of a wearable device (e.g., referring to FIGS. 5-8) that determine an intensity adjustment of a first wavelength light according to an intensity difference of the first wavelength light detected by different light sensors, and determine an adjusting direction (increasing or decreasing) of the first wavelength light according to an intensity variation with time of the first wavelength light detected by the same light sensor so as to alleviate interference from motion artifacts and improve the accuracy of physiological detection.

Although the disclosure has been explained in relation to its preferred embodiment, it is not used to limit the disclosure. It is to be understood that many other possible modifications and variations can be made by those skilled in the art without departing from the spirit and scope of the disclosure as hereinafter claimed.

1. A physiological detection device, comprising:
a first light source, configured to emit light toward a skin surface via an optical element;
a second light source, configured to emit light toward the skin surface;
a first light sensor, configured to receive a first intensity light associated with the first light source and a second intensity light associated with the second light source from the skin surface to generate a first detection data;
a second light sensor, configured to receive a third intensity light associated with the first light source and the second intensity light associated with the second light source from the skin surface to generate a second detection data, wherein the optical element causes the third intensity light to be lower than the first intensity light; and
a processor, configured to adjust a first emission intensity of the first light source and fix a second emission intensity of the second light source.

2. The physiological detection device as claimed in claim 1, wherein the optical element is a view control film.

3. The physiological detection device as claimed in claim 1, wherein
the first light source is an infrared light source, and
the second light source is a green light source.

4. The physiological detection device as claimed in claim 3, wherein the first emission intensity is lower than 10% of the second emission intensity.

5. The physiological detection device as claimed in claim 3, wherein the first intensity light is stronger than the third intensity light by more than two times.

6. The physiological detection device as claimed in claim 1, wherein the processor is configured to
determine an adjustment of the first emission intensity according to an intensity difference between the first detection data and the second detection data, and
determine an adjusting direction of the first emission intensity according to an intensity variation of the first detection data.

7. The physiological detection device as claimed in claim 6, wherein the processor is configured to
reduce the first emission intensity by the adjustment upon the intensity variation being larger than or equal to a positive threshold,
increase the first emission intensity by the adjustment upon the intensity variation being smaller than or equal to a negative threshold, and
stop adjusting the first emission intensity upon the intensity variation being between the positive threshold and the negative threshold.

8. The physiological detection device as claimed in claim 1, wherein the processor is further configured to generate a photoplethysmography signal according to the first detection data without according to the second detection data.

9. The physiological detection device as claimed in claim 1, wherein the first light source and the second light source are configured to emit light simultaneously.

10. A physiological detection device, comprising:
a first light source, configured to emit light toward a skin surface;
a second light source, configured to emit light toward the skin surface;
a first light sensor, configured to receive outgoing light from the skin surface to generate a first detection data; and
a second light sensor, configured to receive outgoing light from the skin surface to generate a second detection data,
wherein the first light source, the second light source, the first light sensor and the second light sensor are arranged to
cause the first light sensor and the second light sensor to receive different intensity of the outgoing light associated with the first light source, and
cause the first light sensor and the second light sensor to receive identical intensity of the outgoing light associated with the second light source.

11. The physiological detection device as claimed in claim 10, wherein the first light source and the second light source are configured to emit light simultaneously.

12. The physiological detection device as claimed in claim 10, wherein
the first light source is an infrared light source, and
the second light source is a green light source.

13. The physiological detection device as claimed in claim 12, wherein a first emission intensity of the first light source is lower than 10% of a second emission intensity of the second light source.

14. The physiological detection device as claimed in claim 12, wherein a light intensity of the outgoing light associated with the first light source received by the first light sensor is more than two times of a light intensity of the outgoing light associated with the first light source received by the second light sensor.

15. The physiological detection device as claimed in claim 12, further comprising a processor configured to generate a photoplethysmography signal according to the first detection data without according to the second detection data.

16. The physiological detection device as claimed in claim 12, further comprising a processor configured to
determine an adjustment of the a first emission intensity of the first light source according to an intensity difference between the first detection data and the second detection data, and
determine an adjusting direction of the first emission intensity according to an intensity variation of the first detection data.

17. The physiological detection device as claimed in claim 16, wherein when the intensity difference is larger, the adjustment is larger.

18. The physiological detection device as claimed in claim 16, wherein
when the intensity variation indicates that the first detection data is increasing, the adjusting direction is to decrease the first emission intensity, and
when the intensity variation indicates that the first detection data is decreasing, the adjusting direction is to increase the first emission intensity.

19. The physiological detection device as claimed in claim 16, wherein the processor is further configured to fix a second emission intensity of the second light source.

20. An operating method of a wearable device, the wearable device being attached to a skin surface and comprising an infrared light source, a green light source, a first light sensor, a second light sensor and a processor, the operating method comprising:
lighting the infrared light source and the green light source;
detecting, using the first light sensor, a first intensity light of the infrared light source and a second intensity light of the green light source to generate a first detection data;
detecting, using the second light sensor, a third intensity light of the infrared light source and the second intensity light of the green light source to generate a second detection data, wherein the third intensity light is lower than the first intensity light; and
adjusting, using the processor, an emission intensity of the infrared light source according to an intensity difference between the first detection data and the second detection data as well as an intensity variation of the first detection data.
